(19) United States
(12) Patent Application Publication
(10) Pub. No.: US 2015/0196275 A1
(43) Pub. Date: Jul. 16, 2015

(54) Ultrasound Measurement Device and Method for Ultrasonic Measurement

(71) Applicant: CyberLogic, Inc., New York City, NY (US)

(72) Inventors: Jonathan J. Kaufman, Brooklyn, NY (US); Gangming Luo, Middle Village, NY (US)

(21) Appl. No.: 14/594,805

(22) Filed: Jan. 12, 2015

Related U.S. Application Data
(60) Provisional application No. 61/927,302, filed on Jan. 14, 2014.

(51) Int. Cl. A61B 8/00; A61B 8/15; A61B 8/08

(52) U.S. Cl. CPC A61B 8/4477; A61B 8/0875; A61B 8/40; A61B 8/5207; A61B 8/15; A61B 8/4209

(57) ABSTRACT

An ultrasound measurement device and related method for ultrasonic measurement are provided. In one embodiment the device includes a first a first ultrasound transducer and a second ultrasound transducer spaced from the first ultrasound transducer and configured to receive a plurality of ultrasound signals transmitted from the first ultrasound transducer through a fixed object. The device further includes means for moving the first and second ultrasound transducers together relative to the object over an area of the object in a first direction, the first ultrasound transducer generating an ultrasound signal of the plurality of ultrasound signals at each of a plurality of locations within the area. In this manner, the device achieves the functionality of array transducers with transducers having fewer transducer elements.

### CROSS-REFERENCE TO RELATED APPLICATIONS

This application claims priority to U.S. Provisional Patent Application No. 61/927,302 filed Jan. 14, 2014, the entire disclosure of which is incorporated herein by reference.

### BACKGROUND OF THE INVENTION

a. Field of the Invention

This invention relates to ultrasound measurement devices and methods and, in particular, to a device and method that achieve the functionality of ultrasound array transducers using transducers having fewer transducer elements.

b. Background Art

Ultrasound measurement devices are used to evaluate a variety of characteristics in both living and non-living things. A conventional ultrasound measurement device includes one more ultrasound transducers that transmit and/or receive ultrasound signals that pass through or are reflected from an object. In order to interrogate a broad area of an object, conventional devices frequently employ one or two-dimensional array transducers. The array transducers include a plurality of transducer elements arranged in a one or two-dimensional array with each element capable of transmitting and/or receiving an ultrasound signal. Although the use of array transducers allows interrogation over a larger area of an object, the transducers are relatively large and costly. Moreover, signal processing for array transducers requires multichannel or multiplexing electronics.

The inventors herein have recognized a need for an ultrasound measurement device and method for ultrasonic measurement that will overcome one or more of the above-identified deficiencies.

### BRIEF SUMMARY OF THE INVENTION

An ultrasound measurement device and method for ultrasonic measurement are provided. In particular, a device and method are provided that achieve the functionality of ultrasound array transducers using transducers having fewer transducer elements.

An ultrasound measurement device in accordance with one embodiment of the invention includes a first ultrasound transducer and a second ultrasound transducer spaced from the first ultrasound transducer. The second ultrasound transducer is configured to receive a plurality of ultrasound signals transmitted from the first ultrasound transducer through a fixed object. The device further includes means for moving the first and second ultrasound transducers together relative to the object over an area of the object in a first direction, the first ultrasound transducer generating an ultrasound signal of the plurality of ultrasound signals at each of a plurality of locations within the area.

An ultrasound measurement device in accordance with another embodiment of the invention includes a first ultrasound transducer configured to transmit a plurality of ultrasound signals into a fixed object and to receive the plurality of ultrasound signals reflected from the object. The device further includes means for moving the first ultrasound transducer relative to the object over an area of the object in a first direction, the first ultrasound transducer generating an ultrasound signal of the plurality of ultrasound signals at each of a plurality of locations within the area.

A method for ultrasonic measurement in accordance with one embodiment of the invention includes the step of positioning first and second ultrasound transducers on opposite sides of a fixed object. The second ultrasound transducer is configured to receive a plurality of ultrasound signals transmitted from the first ultrasound transducer through the fixed object. The method further includes the steps of moving the first and second ultrasound transducers together relative to the object in a first direction to a first location, transmitting a first ultrasound signal from the first ultrasound transducer to the second ultrasound transducer through the object and repeating the moving and transmitting steps over an area of the object.

A method for ultrasonic measurement in accordance with another embodiment of the invention includes the step of positioning a first ultrasound transducer relative to a fixed object. The first ultrasound transducer is configured to transmit a plurality of ultrasound signals into a fixed object and to receive the plurality of ultrasound signals reflected from the object. The method further includes the steps of moving the first ultrasound transducer relative to the object in a first direction to a first location, transmitting a first ultrasound signal from the first ultrasound transducer to the object, receiving a reflection of the first ultrasound signal from the object, and repeating the moving, transmitting and receiving steps over an area of the object.

An ultrasound measurement device and method for ultrasonic measurement in accordance with the present teachings are advantageous relative to conventional devices and methods because the inventive device and method achieve the functionality of array transducers using transducers having fewer transducer elements. As a result, the inventive device and method can be implemented using small and less costly transducers and without multichannel or multiplexing signal processing electronics.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a diagrammatic view of an ultrasound measurement device in accordance with one embodiment of the present teachings.

FIG. 2 is a perspective view of an ultrasound measurement device in accordance with one embodiment of the present teachings.

FIG. 3 is a plan view of a portion of the device shown in FIG. 2.

FIG. 4 is a diagrammatic view of an ultrasound measurement device in accordance with another embodiment of the present teachings.

### DETAILED DESCRIPTION OF EMBODIMENTS OF THE INVENTION

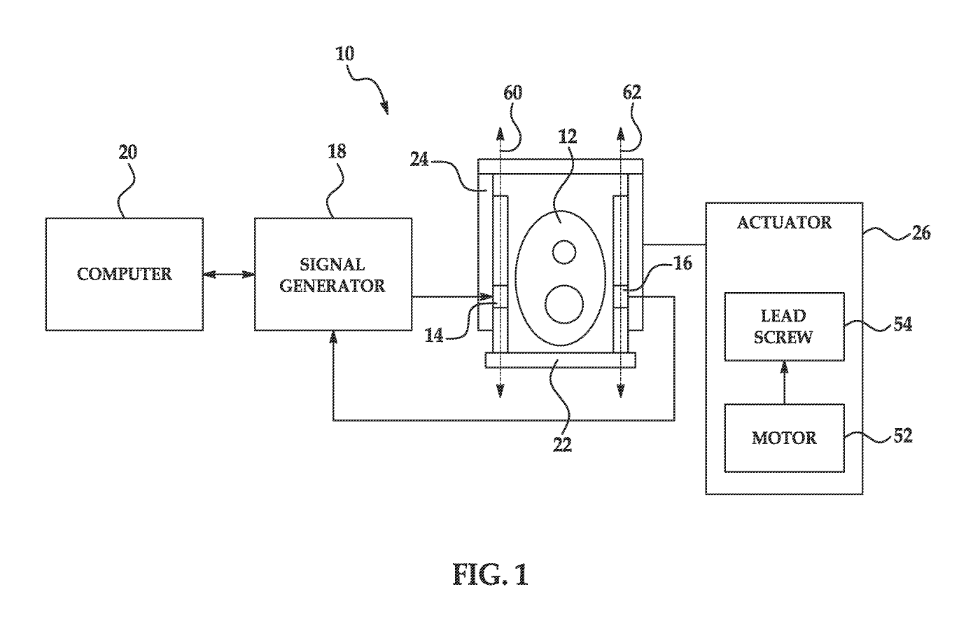

Referring now to the drawings wherein like reference numerals are used to identify identical components in the various views, FIG. 1 illustrates one embodiment of an ultrasound measurement device 10 in accordance with the present teachings. Device 10 is provided to measure one or more characteristics of an object 12. In one embodiment, object 12 comprises a bone such as a radius bone or femoral bone in a living being and device 10 is used to measure a characteristic of the bone such as bone-mineral density, bone mass, cortical thickness, cross-sectional area, medullar thickness, bone width, bone geometry, bone strength or bone fracture risk. It should be understood, however, that device 10 could be used to measure a variety of characteristics for a variety of objects including both living and non-living objects. Device 10 may include ultrasound transducers 14, 16, a signal generator 18, and a computer 20. Device 10 may further include means, such as structures 22, 24 and linear actuator 26, for moving transducers 14, 16 together relative to object 12 over an area of object 12 in one or more directions.

Transducers 14, 16 are provided to transmit and receive, respectively, ultrasound signals. Transducers 14, 16 may comprise immersion transducers (single element longitudinal wave transducers with a ¼ wavelength layer acoustically matched to water) and specifically the 0.25 inch diameter, 2.25 MHz transducers offered for sale by Olympus Corporation of Tokyo, Japan under Model No. U8423047. Transducers 14, 16 preferably each include a single transducer element. Transducers 14, 16 are supported by and mounted within structure 24 and spaced from one another and are movable relative to object 12 as discussed hereinbelow. In the illustrated embodiment, transducer 14 is configured to transmit ultrasound signals while transducer 16 is configured to receive ultrasound signals transmitted by transducer 14 through object 12. It should be understood, however, that transducers 14, 16 are each capable of transmitting and receiving ultrasound signals.

Signal generator 18 is provided to control generation of ultrasound signals from transducer 14 and to process signals received from transducer 16. Signal generator 18 may include a transmitter, receiver and signal processing circuitry and may comprise the signal generator sold under the name “US-KEY” by Lecoeur Electronique Company of Chelles, France. Signal generator 18 powers transducer 14 with an electronic pulse and digitizes the received waveform from transducer 16 before transferring data to computer 20.

Computer 20 is provided to process data received from signal generator 18 and provide information regarding the measured characteristics to a user. Computer 20 may comprise any of a number of conventional computing devices including desktop, laptop or tablet computers or handheld devices such as smartphones. Computer 20 may include a central processing unit (CPU), memory and input/output devices (e.g., a keyboard, mouse, display, etc.) and may be configured to operate sets of executable instructions or code (i.e. software) to analyze data received from signal generator 18 and translate that data for a user. Computer 20 may be configured to process the data received from signal generator 18 in a variety of clinically useful ways including by calculation of the net time delay and mean time duration parameters previously obtained using array transducers as described in U.S. Pat. No. 7,862,510 issued on Jan. 4, 2011, the entire disclosure of which is incorporated herein by reference.

Structure 22, 24 and linear actuator 26 provide a means for moving transducers 14, 16 together relative to object 12 over an area of object 12 in one or more directions. Although an exemplary moving means is illustrated herein, it should be understood that equivalent moving means would encompass a variety of structures having a component whose position is fixed, another component that is movable relative to the fixed component and configured to mount transducers 14, 16, and a device (e.g. a spring) capable of causing movement of the movable component relative to the fixed component.

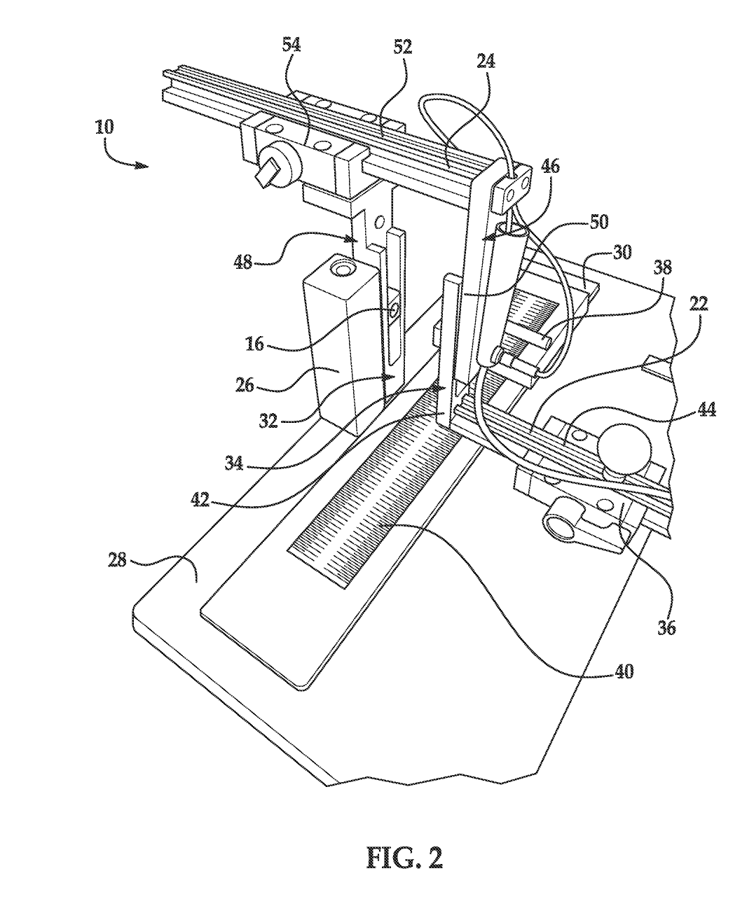

Structure 22 is configured for positioning at a fixed location relative to object 12 and is provided to position and orient object 12. Referring to FIG. 2, structure 22 may include a table 28, a support 30, first and second arms 32, 34 and means 36 for adjusting the position of arm 34 relative to arm 32 and object 12.

Table 28 provides structural support for the other components of structure 22 and a means for orienting those components relative to one another. Support 30 may comprise a transparent block that is movable relative to table 28. Support 30 may further include a transparent rod 38 disposed on the block such that, when device 10 is used to measure a characteristic of a forearm bone, the ulna styloid is placed against the rod 38 to position the forearm. Movement of support 30 enables device 10 to account for both variation in forearm length and the amount of the forearm where the region of interest is located. The transparency of support 30 permits use of a scale 40 disposed on table 28 and underneath support 30 to position the transducers 14, 16 at the clinically recognized “⅓” position—a position along the forearm relative to the ulna styloid that is one third of the length of the forearm (e.g., for a forearm length of 27 cm, the transducers are positioned 9 cm from the distal end of the ulna styloid or 9 cm from the rod 38 on support 30). In this manner, device 10 enables repeated testing of individual patients and among multiple patients at a consistent location. Additional information regarding this aspect of device 10 may be found in copending U.S. patent application Ser. No. 13/201,156 filed Aug. 11, 2001, the entire disclosure of which is incorporated herein by reference. It should be understood that device 10 may be used to take measurements of the forearm bones at positions other than ⅓ position including, for example, the ultras-distal location in the radius bone.

Arms 32, 34 are provided to locate and maintain the location of object 12 and further provide a supporting framework for structure 24 and transducers 14, 16. Arm 32 may be fixed on one side of scale 40. Arm 34 is aligned with arm 32 and movable towards and away from arm 32 such that arms 32, 34 are configured to clamp object 12 between them and fix the position of object 12. Arms 32, 34 make the opposing surfaces of object 12 plane-parallel to one another which is beneficial for the kind of ultrasound propagation used in device 10. Arms 32, 34 also bear the majority of the stress as the arms 32, 34 are closed down on object 12 and, as a result, relatively little stress is exerted on structure 24 supporting transducers 14, 16. Arm 34 may be generally L-shaped with a vertical portion 42 aligned with arm 32 and a horizontal portion 44 that engages position adjustment means 36. Arms 32, 34 each define a vertically extending groove configured to receive a corresponding transducer 16, 14 and tracks along the surface of the grooves along which transducers 14, 16 may move relative to arms 32, 34. To prevent rotation of transducers 14, 16 within the grooves, transducers 14, 16 may extend through apertures in rectilinear blocks coupled to one of structures 22, 24 and configured to be received within the grooves defined in arms 32, 34.

Position adjustment means 36 may comprise a linear guide and, in particular, the linear guide sold under the trademark “DRYLIN T” by iGUS, Inc. of East Providence, R.I., United States of America. Arm 34 may be slid towards and away from arm 32 within the linear guide and locked in place using a locking lever disposed on one side of the linear guide.

Structure 24 is provided to mount transducers 14, 16 and enable movement of transducers 14, 16 relative to object 12. Structure 24 may be configured for movement relative to structure 22 and may include a pair of arms 46, 48 with each arm 46, 48 supporting one of transducers 14, 16. Arm 46 may be generally L-shaped with a vertical portion 50 configured to mount transducer 14. Portion 50 may be aligned with portion 42 of arm 34 of structure 22 in such a way that transducer 14 extends into the groove in arm 34. Arm 46 may further include a horizontal portion 52 extending from one end of portion 50. Arm 48 is configured to mount transducer 16 and may be aligned with arm 32 of structure 22 in such a way that transducer 16 extends into the groove in arm 32. Arm 48 may further include a bracket 54 at one end configured to receive horizontal portion 52 of arm 46 Bracket 54 is configured for movement along portion 52 to move arm 48 towards and away from arm 46 and adjust the spacing therebetween. The spacing between arms 46, 48 is dependent on the spacing between arms 32, 34 of structure 22.

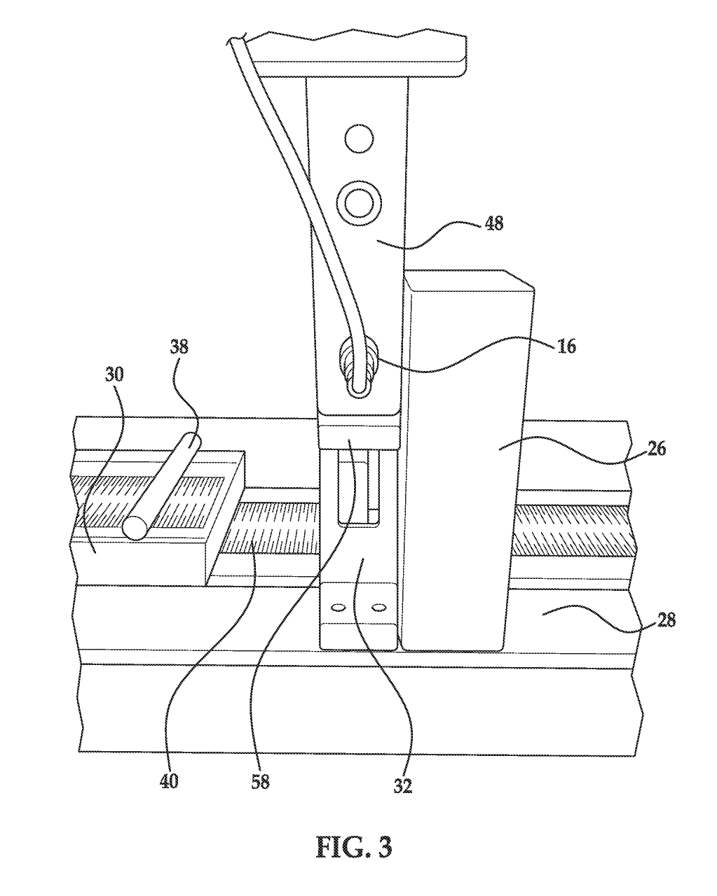

Linear actuator 26 provides a means for moving structure 24 relative to structure 22. Actuator 26 controls movement of structure 24 relative to structure 22 and, therefore, the position of transducers 14, 16 relative to object 12. Referring to FIG. 1, actuator 26 may include a motor 54 and a lead screw 56 rotation of which is controlled by motor 54. Referring to FIG. 3, lead screw 56 may be coupled fixture 24. In particular, lead screw 56 may be coupled to a ledge 58 supporting arm 48 of structure 24. Referring to FIG. 1, rotation of lead screw 56 causes linear motion of ledge 58, structure 24 and transducers 14, 16 along parallel axes 60, 62. Although the illustrated embodiment results in movement of the entire structure 24 relative to structure 22, it should be understood that elements of structure 24 could be stationary once aligned with structure 22 and that transducers 14, 16 could be mounted in such a way that transducers 14, 16 move relative to components of structure 24.

A method for ultrasonic measurement in accordance with one embodiment of the invention may begin with the step of positioning ultrasound transducers 14, 16 on opposite sides of a fixed object 12 such as a radius bone of a forearm or a femoral bone of a leg. The positioning step may begin with the substep of locating structure 22 at a fixed location relative to object 12. Object 12 may be placed on table 28 between arms 32, 34. In the illustrated embodiment, a patient's forearm is positioned lengthwise over scale 40 and the patient's wrist is disposed on support 30 such that the ulna styloid engages rod 38 of support 30 in order to properly position the forearm for measurement at a precise location of the radius bone. Position adjustment means 36 is used to move arm 34 of structure 22 towards arm 32 of structure 22 until object 12 is clamped between arms 32, 34 and the position of object 12 is fixed.

The positioning step may further include the substep of locating structure 24 relative to structure 22. Structure 24 may be positioned such that arm 48 is supported on ledge 58 and arms 46, 48 are aligned with and adjacent to arms 32, 34 of structure 22. Structure 24 is initially located relative to structure 22 such that one of arms 46, 48 is positioned adjacent to a corresponding arm 34, 32, respectively, of structure 22 in such a manner that the corresponding transducer 14, 16 extends through groove in the corresponding arm 34, 32 and is proximate object 12. The other arm 46, 48 is then moved towards the previously positioned arm 46, 48 until the other arm 46, 48 is adjacent the corresponding arm 34, 32 in structure 22 and the transducer 14, 16 extends through the groove in that arm 34, 32.

The method may continue with the step of moving ultrasound transducers 14, 16 together relative to object 12 in a first direction to a first location. In the illustrated embodiment linear actuator 26 causes movement of structure 24 and, therefore, transducers 14, 16, along parallel axes 60, 62 to position transducers 14, 16 at a location. The method may continue with the step of transmitting an ultrasound signal from transducer 14 to transducer 16 through object 12 at that location. The method may continue with the step of repeating the moving and transmitting steps over an area of object 12. In particular, actuator 26 may continue to move structure 24 upward or downward in the illustrated embodiment such that transducers 14, 16 are moved through a series of linearly aligned locations and ultrasound signals are therefore transmitted through a defined area of object 12. In this manner, device 10 achieves the functionality of a device employing one or more linear array transducers while using a single element transducer. In an alternative embodiment, arms 32, 34 of structure 22 may be configured such that arms 32, 34 include grooves permitting two-dimensional movement of transducers 14, 16. In this configuration, the method may further include the step of moving transducers 14, 16 together relative to object 12 in a second direction which may be perpendicular to the first direction. In this manner, device 10 further achieves the functionality of a device employing one or more two-dimensional array transducers while using a single element transducer.

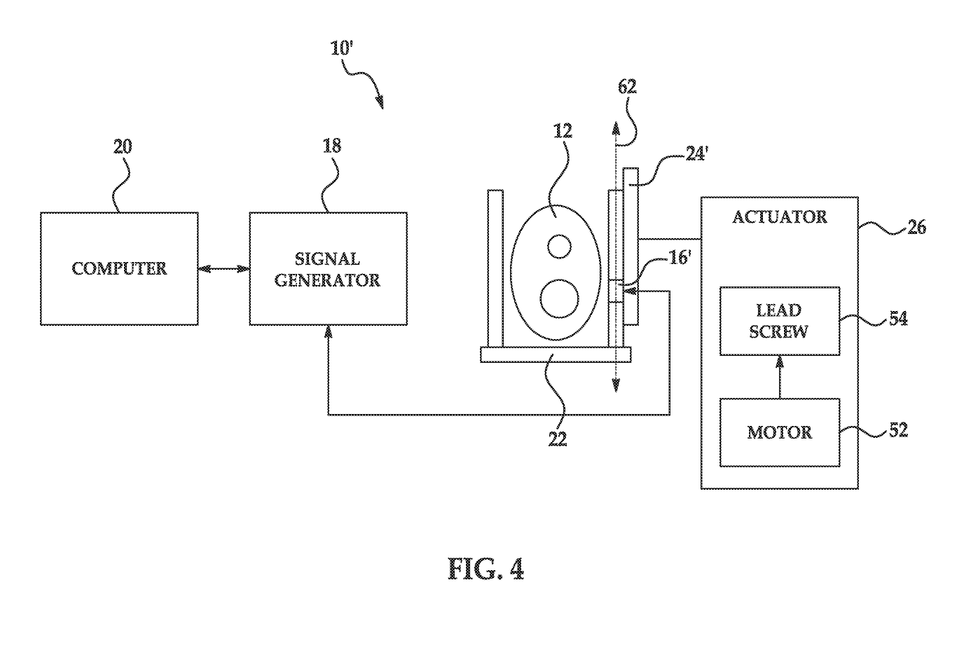

FIGS. 1-3 illustrate a through transmission ultrasound measurement device 10. Referring to FIG. 4, in an alternate embodiment, a device 10′ may instead rely on pulse echo ultrasound measurements. Device 10′ is substantially similar to device 10 and similar components therefore bear the same reference numbers. Device 10′ differs from device 10 in that a simplified structure 24′ replaces structure 24 in device 10. Structure 24′ consists of a single arm 48 mounting a single transducer 16′. Transducer 16′ is configured to both transmit a plurality of ultrasound signals into object 12 and receive the plurality of ultrasound signals as reflected from object 12. Structure 24′, together with structure 22 and actuator 26, provide a means for moving transducer 16 relative to object 12 over an area of object 12 in one or more directions with transducer 16 generating ultrasound signals at each of a plurality of locations within the area.

A method for ultrasonic measurement in accordance with another embodiment of the invention and employing a device such as device 10′ may begin with the step of positioning ultrasound transducer 16′ relative to a fixed object 12 such as a radius bone of a forearm or a femoral bone of a leg. The positioning step may again begin with the substep of locating structure 22 at a fixed location relative to object 12. Object 12 may be placed on table 28 between arms 32, 34. In the illustrated embodiment, a patient's forearm is positioned lengthwise over scale 40 and the patient's wrist is disposed on support 30 such that the ulna styloid engages rod 38 of support 30 in order to properly position the forearm for measurement at a precise location of the radius bone. Position adjustment means 36 is used to move arm 34 towards arm 32 until object 12 is clamped between arms 32, 34 and the position of object 12 is fixed.

The positioning step may further include the substep of locating structure 24′ relative to structure 22. Structure 24′ may be positioned such that arm 48 is supported on ledge 58 and arm 48 is aligned with and adjacent to arm 32 of structure 22 with transducer 16′ extending through a groove in arm 32 and proximate object 12.

The method may continue with the step of moving ultrasound transducer 16′ relative to object 12 in a first direction to a first location. In the illustrated embodiment linear actuator 26 causes movement of structure 24′ and, therefore, transducer 16′, along axis 62 to position transducer 16′ at a location. The method may continue with the steps of transmitting an ultrasound signal from transducer 16′ to object 12 at that location and receiving a reflection of the signal from object 12. The method may continue with the step of repeating the moving, transmitting and receiving steps over an area of object 12. In particular, actuator 26 may continue to move structure 24′ upward or downward in the illustrated embodiment such that transducer 16′ is moved through a series of linearly aligned locations and ultrasound signals are therefore transmitted to object 12 over a defined area of object 12. In this manner, device 10′ achieves the functionality of a device employing one or more linear array transducers while using a single element transducer. In an alternative embodiment, arm 32 of structure 22 may again be configured such that arm 32 include a groove permitting two-dimensional movement of transducer 16′. In this configuration, the method may further include the step of moving transducer 16′ relative to object 12 in a second direction which may be perpendicular to the first direction. In this manner, device 10′ further achieves the functionality of a device employing one or more two-dimensional array transducers while using a single element transducer.

An ultrasound measurement device and method for ultrasonic measurement in accordance with the present teachings are advantageous relative to conventional devices and methods because the inventive device 10 or 10′ and method achieve the functionality of array transducers using transducers 14, 16 or 16′ having fewer transducer elements. As a result, the inventive device 10 or 10′ and method can be implemented using small and less costly transducers and without multichannel or multiplexing signal processing electronics.

While the invention has been shown and described with reference to one or more particular embodiments thereof, it will be understood by those of skill in the art that various changes and modifications can be made without departing from the spirit and scope of the invention.

We claim:

1. An ultrasound measurement device, comprising:
a first ultrasound transducer;
a second ultrasound transducer spaced from said first ultrasound transducer and configured to receive a plurality of ultrasound signals transmitted from said first ultrasound transducer through a fixed object;
means for moving said first and second ultrasound transducers together relative to said object over an area of said object in a first direction, said first ultrasound transducer generating an ultrasound signal of said plurality of ultrasound signals at each of a plurality of locations within said area.

2. The ultrasound measurement device of claim 1 wherein said means for moving said first and second ultrasound transducers includes:
a first structure configured for positioning at a fixed location relative to said object;
a second structure configured for movement relative to said first structure, said first and second ultrasound transducers mounted on said second structure; and,
means for moving said second structure relative to said first structure.

3. The ultrasound measurement device of claim 2 wherein said first structure defines first and second tracks along which said first and second ultrasound transducers are configured to move in said first direction.

4. The ultrasound measurement device of claim 2 wherein said first structure is configured to clamp said object.

5. The ultrasound measurement device of claim 4 wherein said first structure establishes two plane-parallel surfaces in said object.

6. The ultrasound measurement device of claim 2 wherein said means for moving said second structure comprises a linear actuator.

7. The ultrasound measurement device of claim 6 wherein said linear actuator comprises:
a motor; and,
a lead screw rotated by said motor, said lead screw coupled to said second fixture.

8. The ultrasound measurement device of claim 1 wherein both of said first and second ultrasound transducers comprise single element transducers.

9. The ultrasound measurement device of claim 1 further comprising means for moving said first and second ultrasound transducers together relative to said object over said area of said object in a second direction.

10. The ultrasound measurement device of claim 1 wherein said second direction is perpendicular to said first direction.

11. The ultrasound measurement device of claim 1 wherein said object comprises a radius bone.

12. The ultrasound measurement device of claim 1 wherein said object comprises a femoral bone.

13. An ultrasound measurement device, comprising:
a first ultrasound transducer configured to transmit a plurality of ultrasound signals into a fixed object and to receive said plurality of ultrasound signals reflected from said object;
means for moving said first ultrasound transducer relative to said object over an area of said object in a first direction, said first ultrasound transducer generating an ultrasound signal of said plurality of ultrasound signals at each of a plurality of locations within said area.

14. The ultrasound measurement device of claim 13 wherein said object comprises a radius bone.

15. The ultrasound measurement device of claim 13 wherein said object comprises a femoral bone.

16. A method for ultrasonic measurement, comprising the steps of:
positioning first and second ultrasound transducers on opposite sides of a fixed object, said second ultrasound transducer configured to receive a plurality of ultrasound signals transmitted from said first ultrasound transducer through said fixed object;
moving said first and second ultrasound transducers together relative to said object in a first direction to a first location
transmitting a first ultrasound signal from said first ultrasound transducer to said second ultrasound transducer through said object;
repeating said moving and transmitting steps over an area of said object.

17. The method of claim 16 wherein said positioning step includes the substeps of:
locating a first structure at a fixed location relative to said object;
locating a second structure relative to said first structure, said first and second ultrasound transducers mounted on said second structure and said second structure configured for movement relative to said first structure.

18. The method of claim 17, wherein said first structure defines first and second tracks along which said first and second ultrasound transducers are configured to move in said first direction.

19. The method of claim 17, further comprising the step of clamping said object with said first structure.

20. The method of claim 19 wherein said clamping step establishes two plane-parallel surfaces in said object.

21. The method of claim 16 wherein both of said first and second ultrasound transducers comprise single element transducers.

22. The method of claim 16 further comprising the step of moving said first and second ultrasound transducers together relative to said object in a second direction to a second location.

23. The method of claim 22 wherein said second direction is perpendicular to said first direction.

24. The method of claim 16 wherein said object comprises a radius bone.

25. The method of claim 16 wherein said object comprises a femoral bone.

26. A method for ultrasonic measurement, comprising the steps of:
positioning a first ultrasound transducer relative to a fixed object, said first ultrasound transducer configured to transmit a plurality of ultrasound signals into a fixed object and to receive said plurality of ultrasound signals reflected from said object;
moving said first ultrasound transducer relative to said object in a first direction to a first location
transmitting a first ultrasound signal from said first ultrasound transducer to said object
receiving a reflection of said first ultrasound signal from said object; and,
repeating said moving, transmitting and receiving steps over an area of said object.

27. The method of claim 26 wherein said object comprises a radius bone.

28. The method of claim 26 wherein said object comprises a femoral bone.
